(19) United States
(12) Patent Application Publication
(10) Pub. No.: US 2016/0000553 A1
(43) Pub. Date: Jan. 7, 2016

(54) THIN FILM VASCULAR STENT FOR ARTERIAL DISEASE

(71) Applicant: THE REGENTS OF THE UNIVERSITY OF CALIFORNIA, Oakland, CA (US)

(72) Inventors: Daniel S. LEVI, Los Angeles, CA (US); Gregory P. CARMAN, Los Angeles, CA (US); Youngjae CHUN, Pittsburgh, PA (US); Allan W. TULLOCH, Los Angeles, CA (US); Colin KEALEY, Los Angeles, CA (US)

(21) Appl. No.: 14/770,058

(22) PCT Filed: Feb. 25, 2014
(86) PCT No.: PCT/US14/18410

§ 371 Date: Aug. 24, 2015

Related U.S. Application Data
(60) Provisional application No. 61/769,042, filed on Feb. 25, 2013.

(51) Int. Cl. A61F 2/07; A61F 2/86; A61F 2/962; A61F 2/844

(52) U.S. Cl. CPC A61F 2/07; A61F 2/844; A61F 2/86; A61F 2/962; A61F 2002/072

(57) ABSTRACT

A thin film nitinol stent cover is provided that includes a plurality of fenestrations. Each fenestration extends in both longitudinal and transverse dimension, wherein the longitudinal and transverse dimensions are both less than or equal to a critical dimension that inhibits muscle cell migration through the fenestrations.

### CROSS-REFERENCE TO RELATED APPLICATIONS

This application claims the benefit of U.S. Provisional Application No. 61/769,042, filed on Feb. 25, 2013. In addition, this application is related to International Application No. PCT/US2010/026430, filed on Mar. 5, 2010, which claims the benefit of U.S. Provisional Application No 61/158,200, filed Mar. 6, 2009 and U.S. Provisional Application No. 61/158,221, filed Mar. 6, 2009. All the foregoing applications are hereby incorporated by reference in their entirety.

### STATEMENT REGARDING FEDERALLY SPONSORED RESEARCH OR DEVELOPMENT

This invention was made with Government support under Grant No. HL099445, awarded by the National Institutes of Health. The Government has certain rights in the invention.

### TECHNICAL FIELD

This invention pertains generally to implantable devices, and more particularly to an implantable medical device, and surface treatments for the same, for treating peripheral artery disease (PAD).

### BACKGROUND

Lower extremity peripheral arterial disease (PAD) is characterized by the accumulation of atherosclerotic plaque in the arteries of the legs. PAD commonly presents with intermittent claudication, which can be lifestyle-limiting, but may also present as chronic or acute limb ischemia and ultimately require amputation. The prevalence of symptomatic PAD increases with age and is as high as 8% of the general population in persons over 70. In 2008, −1 million endovascular procedures for PAD were performed in the United States, representing a 5-fold increase from a decade earlier and −70% of the total PAD interventions. Due to the aging of our population, endovascular procedures to treat PAD are increasing, with an estimated 2 million procedures performed annually by 2020. Unfortunately, current endovascular treatments are often associated with poor outcomes and new endovascular devices need to be considered for the aging population

One endovascular device for treating PAD is the Viabahn endoprosthesis from Gore. This device uses a self-expandable Nitinol stent backbone lined circumferentially with an expandable polytetrafluoroethylene (ePTFE) liner that is approximately 150 μm in thickness. Data on this device indicate that 1 year primary patency rates for superficial femoral artery disease are between 65-85%. In order to understand the relatively high failure rate of these devices, investigators have identified three key problems. These problems are as follows: First, stenosis tends to occur at the proximal and distal ends of the device. Second, patency rates are independent of treated lesion length. Third a significant percentage of patients (−40% after 5 years) experience late-term thrombosis.

Many of the vessels subject to PAD are relatively small such as having a diameter of just a few millimeters. The relatively thick (−150 μm) ePTFE lining is thus appreciable with regard to such vessel lumens. The resulting restriction in vessel lumen diameter by 300 microns causes or exacerbates proximal and distal restenosis. The impermeability of the ePTFE lining is another issue. Because ePTFE is a thick polymer, it is an impermeable barrier to cell growth and migration. FIG. 1 shows images taken of a stented sheep iliac artery three months after stenting. Due to the image magnification, only two struts 10 of a stent 5 are shown adjacent the iliac artery lining 12. An ePTFE stent liner 12 is on the luminal side of struts 10. Struts 10 are thus abluminal with regard to ePTFE stent liner 12. Given the impermeability of ePTFE stent liner 12, intercellular communication between any luminal endothelial layer on a luminal surface 14 of ePTFE stent liner 12 and an artery wall 15 is impossible. Luminal surface 14 is thus chronically exposed and acellular. This endothelialization failure on luminal surface 14 greatly increases the risk of thrombosis. In contrast, a neointima 11 has proliferated around struts 10. A large body of evidence has shown that intercellular communication between a luminal endothelial monolayer and the underlying vessel wall is critical for preventing excessive smooth muscle cell proliferation leading to neointimal hyperplasia (NIH) and eventually stenosis. Although the impermeability of ePTFE stent liner 12 prevents neointima 11 from invading the vessel lumen, the proliferation of neointima 11 on struts 10 is undesirable and increases the probability of restenosis. The relatively thick and impermeable ePTFE barrier, while preventing smooth muscle cell proliferation (i.e. a beneficial attribute), also prevents nutrient exchange and paracrine communication between intima and media that are key features of normal vessel physiology. Thus, a thick impermeable ePTFE covering is a poor choice for an endovascular device where the goal is to establish non-pathologic vascular homeostasis as quickly and efficiently as possible.

Accordingly, there is a need in the art for improved techniques and devices with regard to preventing restenosis and thrombosis in stented vessels.

### BRIEF SUMMARY OF THE INVENTION

A stent cover is provided that inhibits smooth muscle cell migration and resulting neointimal hyperplasia while promoting a healthy luminal endothelial lining. The stent cover comprises micro-patterned-thin-film nitinol (MTFN) forming a cylinder for enclosing and covering stent struts or truss members. The micro-pattern comprises a plurality of fenestrations in the thin-film nitinol that are large enough to allow sufficient intercellular communication yet are small enough to inhibit neointimal hyperplasia. The stent cover extends in a longitudinal dimension from a proximal end to a distal end. There is a corresponding longitudinal dimension or extent across each fenestration. In that regard, the blood flow within the stented vessel flows generally in the longitudinal dimension. Similarly, there is a transverse dimension or extent across each fenestration that is orthogonal to the longitudinal dimension. These dimensions exist whether each fenestration comprises a similar polygon or are instead irregular. Regardless of the fenestration geometry, the transverse and longitudinal dimensions for each fenestration do not exceed a critical dimension so as to inhibit neointimal hyperplasia. This maximum or critical dimension is comparable to the dimensions of a smooth muscle cell. In one embodiment, the maximum dimension is 10 microns. More generally, the maximum dimension is that which prevents or at least substantially inhibits migration of smooth muscle cells through the fenestrations such as 25 microns or less.

The micro-patterned thin film stent cover is quite advantageous as compared to conventional ePTFE barriers. For example, the fenestrations promote endothelialization on the luminal surface of the stent cover. In contrast to the conventional ePTFE barrier, which lacks such endothelialization, the micro-patterned-thin-film stent cover thus inhibits thrombosis. Although the fenestrations enable endothelialization of the luminal surface and thus inhibit thrombosis, the fenestrations also prevent neointimal hyperplasia on the stent cover luminal surface because the fenestration dimensions are too small to permit smooth muscle cell migration through the fenestrations. In addition, the resulting cellular communication between the endothelial lining on the stent cover luminal surface and the vessel wall adjacent to the stent cover abluminal surface is believed to inhibit hyperplasia on the abluminal surface of the stent cover. In contrast, the neointimal proliferation (neointimal 11 of FIG. 1) on the abluminal surface of conventional ePTFE barriers is plainly undesirable. Furthermore, the micro-patterned thin-film stent cover is markedly thinner than conventional ePTFE barriers and thus resists restenosis resulting from flow constrictions. These and other advantages of the advantageous micro-patterned-thin-film stent cover disclosed herein may be better appreciated from the following detailed description without placing limitations thereon.

### BRIEF DESCRIPTION OF THE DRAWINGS

The invention will be more fully understood by reference to the following drawings which are for illustrative purposes only:

FIG. 1 shows an image taken from an ePTFE covered stent after 3 months in a sheep iliac artery.

FIG. 2A illustrates an assembled view of a PAD stent in accordance with the present invention.

FIG. 2B illustrates an exploded view of the PAD stent of FIG. 1.

FIG. 3A shows a schematic diagram of an implanted stent with MTFN covering of the present invention and its effect on “edge effect” stenosis.

FIG. 3B shows a schematic diagram of an implanted stent with ePTFE covering and its effect on “edge effect” stenosis.

FIG. 4 shows a schematic diagram of an implanted stent with MTFN covering of the present invention and effect on SMC (smooth muscle cell) migration.

FIG. 5 shows a schematic diagram of an implanted stent with MTFN covering of the present invention and resulting endothelial monolayer.

FIG. 6 is a graph showing the influence of treatment time on the wetting angle of the MTFN film of the present invention.

FIG. 7 shows SEM images of four MTFN sheets, each with different micro patterns of fenestrations in accordance with the present invention.

FIG. 8A and FIG. 8B show optical microscopy images of two films having diamond pattern fenestrations with dimensions of 7.5 μm×10 μm and 45 μm×60 μm, respectively.

FIG. 9A through FIG. 9C show molecular analysis of the hemocompatibility of TFN as compared to ePTFE, with FIG. 9A showing total thrombus and FIGS. 9B and 9C showing fibrin and platelet deposition, respectively.

FIG. 10A through FIG. 10C show images of the effects of surface wettability and the endothelial monolayer in vitro after 1 week for contact angles of 0°, 40°, and 65°, respectively.

FIG. 11 shows a representative image of a vessel wall treated with the stent of FIG. 8A having 7.5 μm×10 μm perforations.

FIG. 12 shows the contralateral iliac artery treated with the stent covering of FIG. 8B having 45 μm×60 μm perforations.

FIG. 13 shows a graph of neointimal area for various fenestration sizes.

FIG. 14A through FIG. 14C show images of the wall of an artery treated with a 45 μm×60 μm stent covering at low magnification, medium magnification, and high magnification respectively.

FIG. 15A shows images of HAECs grown on un-patterned TFN.

FIG. 15B (scale bar 100 μm) and FIG. 15C (scale bar 50 μm) show HAECs grown on MTFN with a lattice pattern.

### DETAILED DESCRIPTION

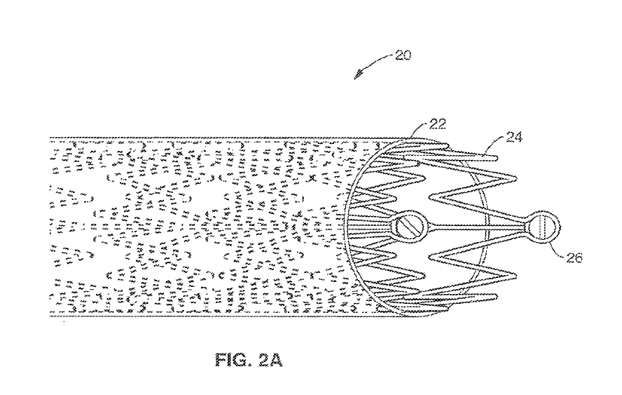

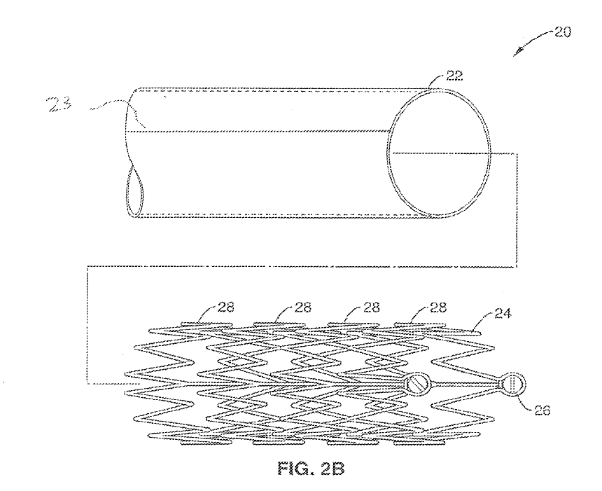

Referring more specifically to the drawings, for illustrative purposes the present invention is embodied in the apparatus generally shown in FIG. 2 through FIG. 15C. It will be appreciated that the apparatus may vary as to configuration and as to details of the parts, and that the method may vary as to the specific steps and sequence, without departing from the basic concepts as disclosed herein.

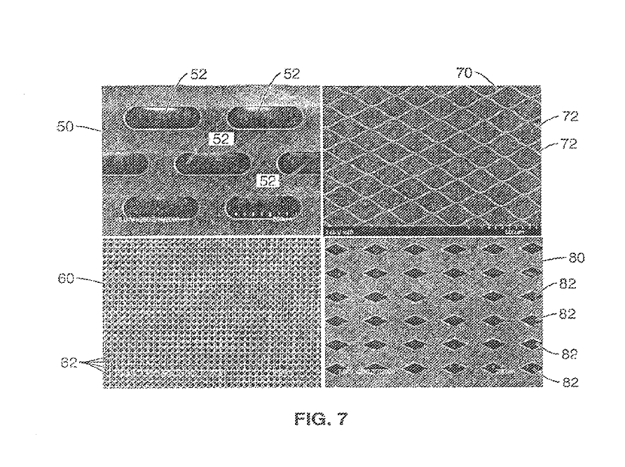

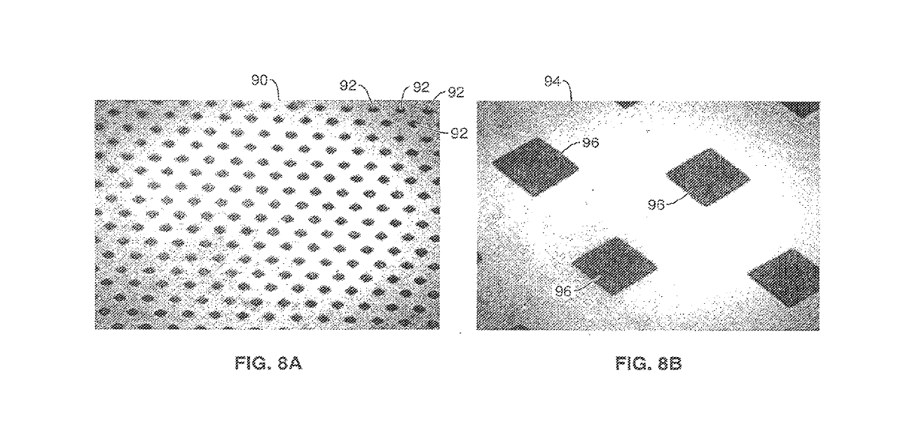

FIG. 2A and FIG. 2B illustrate assembled and exploded views (respectively) of PAD stent 20 in accordance with the present invention. Stent 20 generally comprises a micro-patterned-thin-film nitinol (MTFN) cover 22 that is disposed around a collapsible truss 24. As shown in FIG. 2B, truss 24 may comprise a plurality of undulating wire segments or stent struts 28 that are coupled to anchor points 26 that allow the truss 24 to be compressed in a collapsed configuration (not shown) to a delivery location. In one embodiment, struts 28 may comprise nitinol. The MTFN sheet 22 generally forms an extremely low profile (e.g. 5 μm thick) tubular structure around the truss 24 and has a plurality of perforations shown in greater detail in FIGS. 7 through 8B.

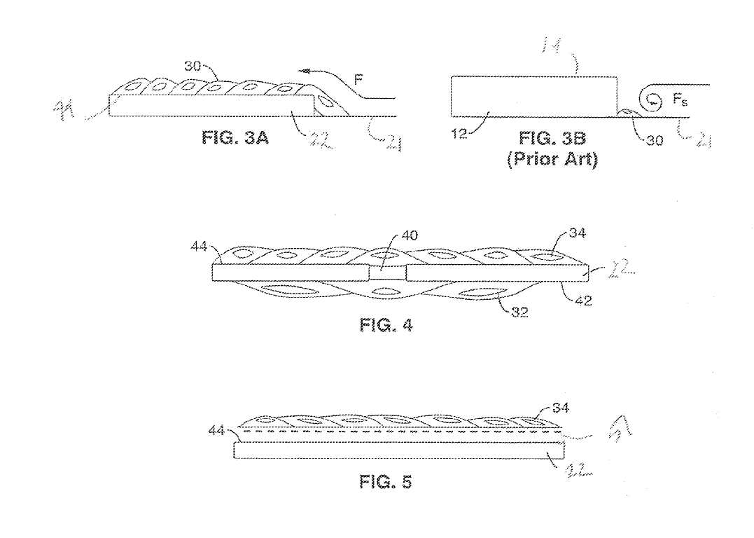

Referring to FIG. 3A through FIG. 5, it will be appreciated that MTFN stent cover 22 has a number of advantages. For example, FIG. 3A shows a portion of an MTFN stent cover 22 contacting a vessel wall 21. For illustration clarity, the stent struts on the luminal side of MTFN stent cover 22 are not illustrated in FIG. 3A. Because stent cover 22 comprises thin-film nitinol, it presents an insignificant barrier to the blood flow direction as indicated by arrow F. This lack of flow restriction inhibits restenosis of the stented vessel. In contrast, the much greater thickness of conventional ePTFE stent liner 12 as shown in FIG. 3B presents a much greater obstruction to blood flow as indicated by flow separation zone Fₛ. In addition, MTFN stent cover 22 promotes endothelialization 30 on its luminal surface 23 to inhibit thrombosis whereas luminal surface 14 of conventional ePTFE stent liner 12 is acellular and thus promotes thrombosis.

An example fenestration 40 in MTFN stent cover 22 is shown in FIG. 4. The longitudinal and transverse dimensions across fenestration 40 are small enough to prevent migration of smooth muscle cells 32 in the stented vessel wall through fenestration 40 (i.e., from an abluminal surface 42 of MTFN stent cover 22 to the luminal surface 44). For example, these dimensions in one embodiment do not exceed 10 microns with 1 micron precision. Thus, endothelial cells 34 on luminal surface 44 do not get invaded by smooth muscle cells 32 such that neointimal hyperplasia is prevented while still allowing for intercellular communication through the graft's thickness.

In addition, the MTFN stent covers disclosed herein may include a surface treatment to increase hydrophilicity. This increased hydrophilicity is illustrated symbolically in FIG. 5 by dotted line 51. The resulting chemical modification of stent surfaces such as luminal surface 44 facilitates growth of a robust endothelial monolayer 34.

MTFN stent cover 22 is processed to specific dimensions and composition to promote adaptation within the patient's body. Nitinol, or Nickel Titanium, is an equiatomic (1 atom Ni, 1 atom Ti) shape memory alloy, and is commonly used in endovascular devices in the form of bulk Nitinol (>100 microns thick). MTFN stent cover 22 of the present invention comprises thin film nitinol (TFN) that is fabricated in sheets approximately 5 μm thick via sputter deposition that has only recently become available for practical uses.

In one embodiment, MTFN stent cover 22 may be generated using a “hot-target” sputter deposition process, detailed in International Application No. PCT/US2010/026430 (the '430 application) that consistently generates thin-film nitinol (TFN) with a <0.5% atomic compositional variation. In addition to its high purity, the TFN produced by this method is both extremely smooth (surface roughness of 5 nm) and strong (tensile strength of 500 MPa).

As discussed in the '430 application, a semiconductor substrate may be patterned using a deep reactive ion etching (DRIE) process to create a patterned substrate. Nitinol is then sputtered onto the patterned substrate. Although the bottoms of the etched trenches in the substrate also receive a sputtered layer of nitinol, those areas are separated from the nitinol deposited on the non-trenched portions of the patterned substrate by the vertical trench walls produced by the DRIE process. When the nitinol film is then released from the patterned substrate, the nitinol film will then have fenestrations corresponding to where the trenches were produced on the patterned substrate. As detailed further in the '430 application, the use of the DRIE-patterned substrate is quite advantageous because of its relatively tight tolerance—for example, the trench shapes (and thus the resulting fenestrations in the patterned thin-film nitinol) may have a tolerance of a 1 micron or less. In contrast, wet etching techniques typically have much coarser tolerances. Since the fenestrations disclosed herein are relatively small (e.g., having longitudinal and transverse dimensions of 10 microns or less), it is advantageous to employ the DRIE process discussed in the '430 application. However, it will be appreciated that nitinol may be sputtered onto an un-patterned substrate such that the fenestrations are subsequently formed using conventional wet-etching techniques in alternative embodiments.

Because the patterned substrate is typically planar, the DRIE process discussed in the '430 application typically produces a planar thin-film nitinol sheet. In contrast, stent cover 22 is cylindrical. To form such a three-dimensional structure from the nitinol sheet, the longitudinal edges of the sheet are sealed together along a seam 23 as shown in FIG. 2B. It will thus be appreciated the stent cover length (the longitudinal extent of a stent cover) depends upon the longitudinal length of the initial thin-film nitinol sheet that is then sealed along its longitudinal edges to form seam 23. Conversely, the initial thin-film nitinol sheet will have some transverse length along its proximal and distal edges. It is this transverse length for the initial thin-film nitinol sheet that determines the stent cover lumen diameter. Alternatively, nitinol could be sputtered onto a patterned tubular mandrel and then released from the mandrel to produce stent cover 22. In such embodiments, there would be no intermediate stage of forming a planar sheet and then sealing the sheet into a tubular structure. To distinguish the sealed stent cover from the initial thin-film sheet, the term “MTFN sheet 22” refers to the initial planar thin-film sheet whereas “MTFN stent cover 22” refers to the stent cover that results from sealing the thin-film sheet along seam 23. In yet additional alternative embodiments, nitinol may be sputtered onto a patterned planar substrate. A sacrificial layer such as a chromium layer may then be deposited along a swath of the sputtered nitinol and then additional nitinol sputtered onto this sacrificial layer and the initially-deposited nitinol outside of the area covered by the sacrificial layer. A “layer cake” nitinol sheet results that has sealed edges (nitinol deposited on nitinol) and also nitinol layers that are separated by the sacrificial layer. This sacrificial layer is exposed in the fenestrations of the nitinol layers and may thus be etched away. A three-dimensional (i.e., tubular) structure results that needs no sealing along any longitudinal edges despite the use of planar substrates. Additional details regarding the manufacture of such a tubular thin-film nitinol structure may be found in U.S. Pat. No. 6,790,298, the contents of which are incorporated by reference in their entirety.

The MTFN stent cover 22 of the present disclosure generally has a thickness of less than 50 microns, and preferably has a thicknesses ranging from about 0.1 microns to about 30 microns. Preferably, the thin films may have a thickness ranging from about 0.1, 1, 2, 4, 5, 10, 15, 20, 25, 30 or 50 microns to about 4, 5, 10, 15, 20, 25, or 30 microns. More preferably, the thin films may have a thickness of from about 4 microns to about 12 microns.

As a result of the relative thinness, covering a stent truss 24 (FIG. 2B) with the thin memory metal film of the present disclosure results in a minimal and inconsequential increase in the size of the overall device. For example, MTFN can be manufactured in films of from about 5 to about 8 μm thickness, so that covering a stent with MTFN adds very little bulk to the devices. The stent struts can have a thickness in the range of, for example, about 2 μm, 4 μm, 6 μm, 7 μm, 10 μm, 17 μm, 18 μm, or 20 μm.

Both truss 24 and MTFN stent cover 22 can be produced in a range of shapes and sizes. For example, thin memory metal alloy films can be made square or rectangular e.g. when laid flat, the sheet can have the appearance of a rectangle with a longer longitudinal dimension and a shorter transverse dimension. Each dimension of such a square or rectangle can be selected from a wide range.

In some embodiments, the width (the transverse dimension) of such a square or rectangle may be in the range of, for example, about 0.5 mm, 1 mm, 3 mm, 5 mm, 10 mm, 16 mm, 20 mm, 25 mm, 30 mm, or 40 mm. The width is generally a function of the internal diameter of the lumen to be treated.

Correspondingly, the length (longitudinal dimension) of such a square or rectangle may be in the range of, for example, about 0.5 mm, 2 mm, 5 mm, 15 mm, 20 mm, 10 mm, 50 mm, or 100 mm. Generally, the length is a function of the size of the region to be treated.

Adjacent sides of sheet 22 need not be perpendicular. The sheet 22 can have a form that is not an endless loop; for example, the sheet can have two distal edges as ends of the sheet, bounding the length dimension.

Thin memory metal alloy films may be made in a wide variety of shapes other than square or rectangular. For example, thin memory metal alloy films may be made to resemble other polygons, circles, ovals, crescents, or an arbitrary shape.

In one embodiment, the sheet 22 comprises a generally rectangular thin film sheet wrapped into a generally tubular shape having a longitudinal and radial direction. The two distal edges of the sheet define two ends of the tubular shape and meet or overlap.

In another embodiment, the sheet has a compacted form with a first internal diameter and a deployed form with a second internal diameter larger than the first internal diameter such that the sheet contacts the lumen wall at a radius equal to or slightly larger than the radius of the lumen.

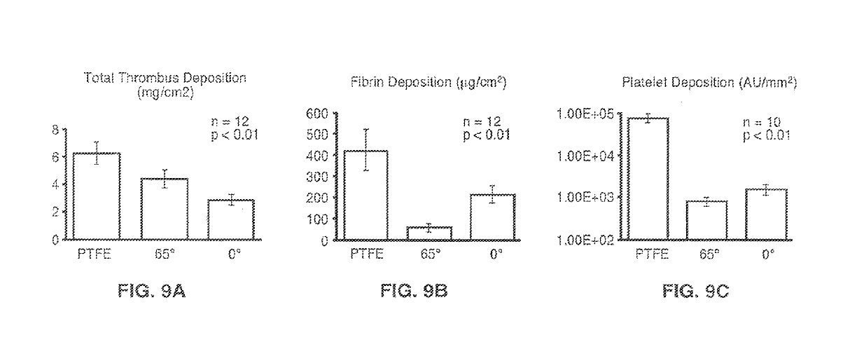

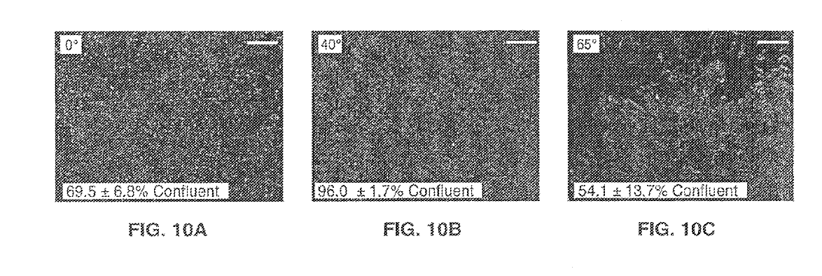

Another advantage of MTFN sheet 22 of the present invention is the ability to control its surface characteristics by chemical treatment. In a preferred embodiment, the MTFN sheet 22 is treated in accordance with the methods disclosed in the '430 application, which includes removal of the film's native surface oxide layer with a buffered oxide etchant, followed by passivation in nitric acid (HNO₃) and submersion in hydrogen peroxide (H₂O₂). This process produces a TiO layer (e.g., 100 nm thick) and allows charged hydroxyl groups to attach to the surface as confirmed with high resolution transmission electron microscopy (HRTEM). The negative charge mimics the negative charge of the vascular endothelium and can be manipulated to facilitate rapid endothelialization (see FIGS. 10A-10C described in further detail below).

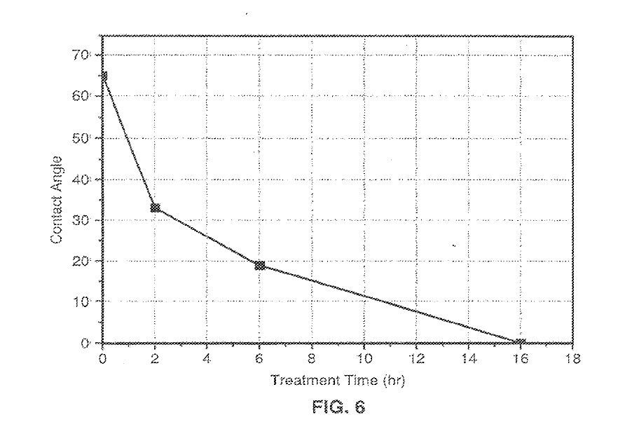

One tool to characterize the hydrophilicity of MTFN sheet 22 surface 44 is wetting angle. FIG. 6 shows the influence of treatment time on the wetting angle of the sheet 22. At the extremes, untreated TFN has a wetting contact angle of 65°, whereas the sheet 22 treated for 15 hours in H₂O₂ has a contact angle of 0° (i.e. a super-hydrophilic surface). This treatment modifies the surface characteristics (i.e. negative charge and TiO layer) to achieve contact angles ranging from 0° to 65°, which can be used to vary the characteristics of the stent 20.

Another significant advantage of MTFN stent cover 22 is the ability to precisely control permeability (i.e. porosity) and geometry. As discussed earlier, the deep reactive ion etching (DRIE) method disclosed in the '430 application may be used to produce relatively small fenestrations with high precision (tolerance of 1 micron or less). Thus, fenestrations having the maximum dimensions of, for example, 25 microns or less, or even 10 microns or less, to inhibit smooth muscle migration through the fenestrations is achievable.

Examples of four different fabricated MTFN sheets are shown in FIG. 7 to illustrate the wide variety of fenestration shapes that may be sized so as to inhibit smooth muscle cell migration yet enable fluid exchange to promote endothelialization. For example, a sheet 50 may comprise a plurality of oval slots 52, a sheet 60 may comprise a pattern of circular holes 62, a sheet 70 may comprise thin diamond-shaped borders 72 separating the fenestrations in a “chain-link fence” fashion, and a sheet 80 may comprise a plurality of diamond-shaped fenestrations 82.

The fenestrations may be aligned in precise regular arrays (i.e. 2 micron resolution or less). This presents a unique advantage of MTFN sheet 22 over ePTFE and other biomaterials. For example, FIG. 8A is an optical microscopy image of a sheet 90 having diamond-shaped fenestrations 92 with a longitudinal dimension of 10 microns across each fenestration 92 and a transverse dimension across each fenestration 92 of 7.5 microns (7.5 μm×10 μm). Similarly, FIG. 8B is an optical microscopy image of a sheet 94 having diamond-shaped fenestrations 96 having dimensions of 45 μm×60 μm. These patterns were used in the stent cover pilot studies discussed below. Each fenestration 92 is smaller than a smooth muscle cell (SMC) 32 (see FIG. 4), allowing the sheet 90 to act as a filter that prevents SMC migration onto the stent luminal surface and the resulting NIH, but still permits exchange of nutrients and cell-to-cell signaling molecules through fenestrations 92. Such aperture dimensions allow biologic interactions to achieve improved outcomes.

The following discussion detail tests performed on the MTFN 22 of the present invention experimentally correlate TFN's wetting contact angle to its hemocompatibility and ability to support endothelial cell growth in vitro as well as neointimal growth in vivo. To demonstrate hemo-compatibility of the MTFN-based stents of the present invention, a series of experiments were conducted. Prototype stents were fabricated using non-micropatterned TFN with the extreme contact angles of 65° or 0°. The resulting devices, along with an ePTFE control, were deployed in a custom in vitro model that circulates fresh whole blood simulating moderate arterial stenosis. Following testing, the three materials were analyzed qualitatively with scanning electron microscopy (SEM) and quantitatively via a series of molecular assays.

FIG. 9A through FIG. 9C are graphs of the results from molecular analysis of the hemocompatibility of TFN as compared to ePTFE. In particular, FIG. 9A shows the total thrombus deposition for the ePTFE control and the two TFN examples whereas FIGS. 9B and 9C show fibrin and platelet deposition, respectively, for those devices. In the test, the devices were placed in a whole blood circulation model at a wall shear rate simulating a moderate arterial stenosis for 3 hours.

Both the 0° and the 65° degree TFN devices demonstrated markedly less blood product as compared to the deposition on the ePTFE control device. In the case of platelets (FIG. 9C), the prototype ePTFE stent had more than two orders of magnitude greater deposition than the prototype TFN devices. Scanning electron microscopy (SEM) and mass spectrometry data confirmed all of these findings. On SEM, the ePTFE device was almost completely obscured by thrombus, while both types of TFN were clearly visible beneath a sparse covering of fibrin, platelets, and RBCs. Likewise, mass spectrometry confirmed the trends measured in FIGS. 9A through FIG. 9C. For example, mass spectrometry analysis demonstrated approximately ten times more deposition of hemoglobin α and β chains on the ePTFE device than on either TFN device. This data strongly suggests that TFN has markedly improved hemocompatibility as compared to ePTFE, and that contact angle exerts significant effects on the interaction with blood. Based on these results, it is believed that MTFN stent 20 of the present disclosure will have a reduced incidence of both acute and late-term thrombosis as compared to ePTFE counterparts.

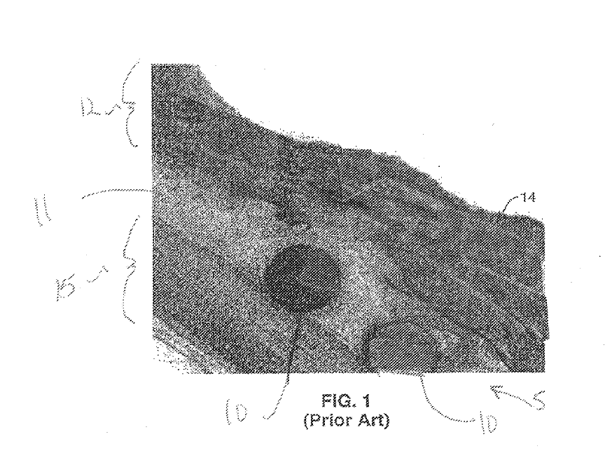

In addition, the MTFN surface wettability was studied for its effects on endothelial growth and neointimal architecture. A primary measure of an indwelling intravascular device's success is its ability to rapidly and completely endothelialize with a minimal amount of neointimal growth. As discussed with regard to FIG. 1, this is a significant limitation of ePTFE covered stents. To evaluate the correlation between contact angle and endothelialization, TFN with 3 different contact angles (0°, 40°, and 65°) was tested. In particular, Human Aortic Endothelial Cells (HAECs, Lonza, Switzerland) were cultured on the TFN samples for 1, 3, and 7 days. After each test time, samples were stained with AlexaFluor 488 phalloidin (f-actin specific) and DAPI (nucleus).

Representative images of the effects of surface wettability and the endothelial monolayer in vitro after 1 week are shown in FIG. 10A through FIG. 10C, (N=3, p<0.05). These results indicate that TFN with a 40° contact angle supports a more confluent, and faster-growing endothelial monolayer than the 65° or 0° films.

In vivo data was also acquired showing the effects of contact angle on the healing response that follows endovascular device placement. For this study, two non-micropatterned TFN covered stents with a contact angle of either 0° or 65° were fabricated and deployed in the iliac arteries of swine. After 30 days, devices were harvested, and sectioned for pathology. The 0° specimen demonstrated a thinner, more organized neointima with less inflammatory infiltrate as compared to the 65° device. This data correlates well with the in vitro results, showing increased endothelial growth on the 0° thin films as compared to the 65° films.

Accordingly, an MTFN sheet 22 may be fabricated according to an optimal contact angle (e.g. below at or 40°) by controlling the processing time (e.g. treatment time within a hydrogen peroxide (H₂O₂) bath (see FIG. 6)) to support rapid growth of a functional endothelial monolayer on the MTFN device 20, while also producing a surface that minimizes thrombus deposition (see FIG. 9A).

Micropattern pore size was also studied with respect to neointimal thickness and abluminal SMC migration. Pilot studies were performed to examine the effects of MTFN perforation size (i.e. permeability) on SMC migration and neointimal growth in vivo. Three types of MTFN sheets were used for this study. Each sheet had diamond-shaped apertures with dimensions of 7.5×10 μm (sheet 90 of FIG. 8A), 10×20 μm, and 45×60 μm (sheet 94 of FIG. 8B). MTFN covered stents were then placed in the iliac arteries of swine and harvested after 30 days.

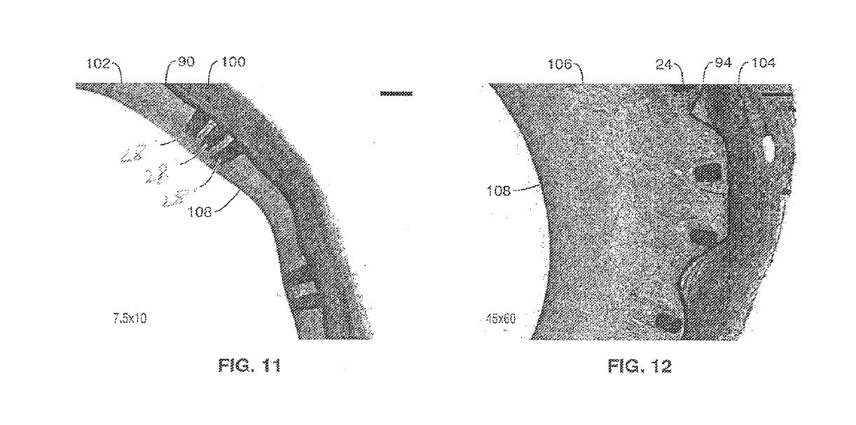

FIG. 11 shows a representative image of a vessel wall 100 treated with an MTFN stent cover 90 having 7.5μm×10 μm perforations (FIG. 8A). The neointima 102 is thin, well-organized, and does not extend into the vessel lumen 108 beyond the level of the stent struts 28. FIG. 12 shows the contralateral iliac artery 104 of the same animal treated with the stent cover 94 of FIG. 8B having 45 μm×60 μm perforations (at the same magnification. The neointima 106 is thick, disorganized, and has increased numbers of inflammatory cells.

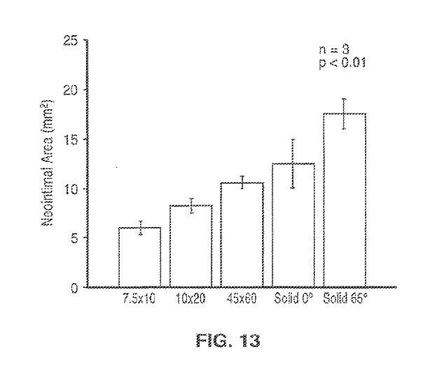

For quantitative comparison, FIG. 13 shows a graph of neointimal area (NIA—defined as the area between the MTFN stent cover and open vessel lumen 108) for various fenestration sizes, ranging from 7.5×10 micron fenestrations to 45×60 microns fenestrations. FIG. 13 shows an increase in NIA with increasing MTFN fenestration size. Note that the NIA (6.0±0.7 mm²) for the 7.5 μm×10 μm device is substantially smaller than the NIA for an ePTFE covered stent (not illustrated), which showed an NIA of 11.9±4.3 mm² after 3 months of implantation in a sheep. On the far right of FIG. 13, NIA measurements are included for two non-micropatterned films with 0° and 65° contact angles (N=1 for the un-patterned devices). These results demonstrate that an absence of fenestrations leads to increased NIA, regardless of contact angle (i.e. similar to the relatively impermeable ePTFE devices), likely from a lack of intercellular communication between the layers of the vessel wall for a rapid, well-organized healing response.

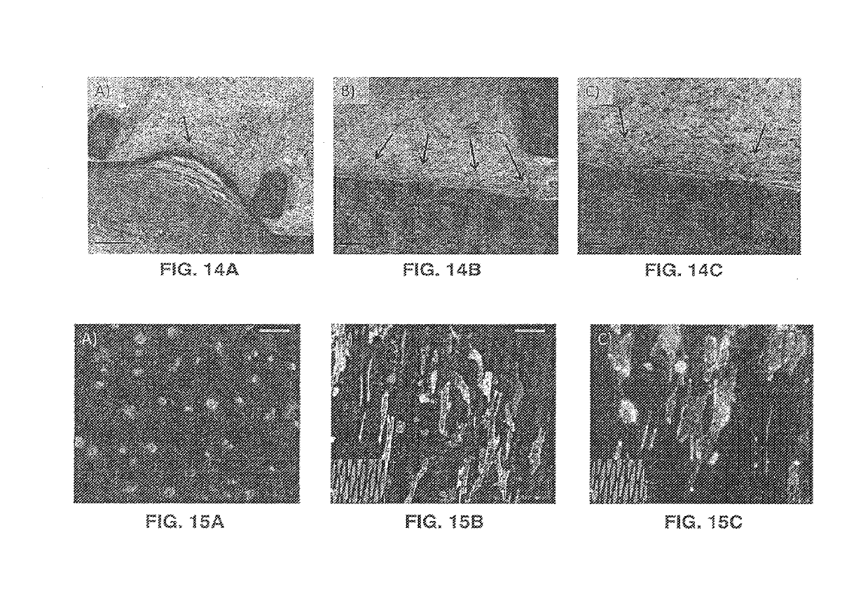

This study demonstrated that SMCs 32 clearly migrate across the MTFN barrier in devices with large fenestrations (e.g. 45 μm×60 μm) but not in devices with small fenestrations (e.g. 7.5 μm×10 μm). FIG. 14A through 14C show the wall of an artery treated with a 45 μm×60 μm device at low magnification, medium magnification, and high magnification, respectively. The fenestrations can be seen as small breaks in the MTFN stent cover. At the site of the fenestrations, robust smooth muscle cell migration is observed (marked by the black arrows, most notably in FIG. 14B and FIG. 14C). These areas of cell migration appear as “mini-volcanoes,” and significant SMC migration from the abluminal side of the MTFN stent covers is observed.

These images provide evidence of a “critical dimension” whereby SMC migration across the MTFN is inhibited while a path for intercellular communication is still in place. Accordingly, the longitudinal and transverse dimensions for the fenestrations are ideally less than 10 μm, and preferably between 5 μm and 10 μm. More generally, these dimensions should be less than 25 microns, and even more generally should be less than or equal to a dimension that inhibits smooth muscle migration.

Based on these in vivo an MTFN covered stent designed with a “critical dimension” for its fenestrations (e.g. less than 10 μm and ideally between about 5 μm and about 10 μm) will reduce NIH, while still providing a channel for communication between an endothelial layer on the device's luminal side with the underlying vessel wall in a manner not possible with ePTFE-based devices.

The effects of micropattern geometry on endothelial growth have also been examined. For these studies, Human Aortic Endothelial Cells (HAECs) were grown on MTFN with different geometries and allowed to proliferate for 3 days. Samples were stained with DAPI and Phalloidin and imaged with a fluorescent microscope. FIG. 15A shows HAECs grown on unpatterned TFN. Their rounded “cobblestone” morphology is typical of ECs grown under normal culture conditions. FIGS. 15B (scale bar 100 μm) and 15C (scale bar 50 μm) show HAECs grown on MTFN with a lattice pattern (similar to MTFN sheet 70 of FIG. 7). The lower-left inset shows the pore geometry used. The cells adopt an elongated morphology that follows the MTFN geometric designs, indicating that micropattern geometry regulates endothelial morphology (e.g. elongate and/or faceted, straight-edged geometry being preferred). Of note, these results were obtained using TFN with a 65° contact angle, which explains why HAEC growth was not confluent given the data presented in the previous section, i.e. a wetting angle of 40° appears to be optimal. Regardless, these findings are significant when one considers the relationship between endothelial morphology and function. It is well established that arterial regions (such as bifurcations) prone to atherosclerosis are exposed to low-oscillating shear stress. ECs in these regions adopt a round, “cobblestone” morphology and have increased expression of immune-regulating surface receptors. Conversely, “atheroprotected” regions are typically straight and exposed to high unidirectional shear stress. ECs found here adopt an elongated “spindle” morphology and have decreased expression of pro-inflammatory surface receptors. Based on these findings, an appropriately patterned MTFN stent 20 of the present invention may serve as a scaffold to encourage an atheroprotective EC morphology in ways not possible with ePTFE-based stents.

The MTFN-based stents of the present invention address two main problems associated with ePTFE covered stents: 1. Patency independent of treated lesion length, and 2. late-term graft thrombosis. Based on the above findings, it is believed that ePTFE's thickness causes a size mismatch with the vessel wall that leads to restenosis, and that the relatively impermeable ePTFE barrier prevents communication between the luminal neointima and abluminal vessel wall. This causes a failure of ePTFE grafts to endothelialize and creates a chronically exposed thrombogenic surface that predisposes patients to late-term thrombosis.

The MTFN-based stent of the present invention overcomes these limitations in at least three ways. First, the ultra-low profile of the MTFN stent 20 of the present invention eliminates edge-effect stenosis and persistent flow separation zones by allowing for proximal and distal cell migration. Second, the MTFN stent 20 of the present invention has a porosity which can be controlled such that abluminal SMC migration is prevented, but still allows for intercellular communication between neointima and vessel wall throughout the length of the stent. Third, the MTFN stent 20 of the present invention has surface characteristics and fenestration geometry that can be optimized to encourage growth of a non-thrombogenic, non-immunogenic endothelial layer on the stent's luminal surface that is in direct communication with the underlying vessel wall to maintain long-term patency.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. An implant for treating peripheral arterial disease, comprising:
a plurality of stent struts; and
a micro-patterned-thin-film (MTFN) nickel titanium (NiTi) stent cover configured to be positioned circumferentially around the plurality of stent struts, the stent cover including a plurality of fenestrations;
wherein the fenestrations are sized to be small enough to substantially inhibit abluminal migration through the fenestrations.

2. An implant as recited in claim 1, wherein the fenestrations are sized to be large enough to allow for intercellular communication through the sheet.

3. An implant as recited in claim 2, wherein the MTFN stent cover extends in a longitudinal dimension, and wherein the fenestrations are sized to have a maximum longitudinal dimension of 10 μm or less, and wherein the fenestrations are sized to have a maximum transverse dimension of 10 μm or less.

4. An implant as recited in claim 2, wherein the longitudinal and transverse dimensions for the fenestrations are between approximately 5 μm and 10 μm.

5. An implant as recited in claim 1, wherein the MTFN stent cover has a thickness of less than approximately 50 μm.

6. An implant as recited in claim 5, wherein the MTFN stent cover has a thickness of less than approximately 10 μm.

7. An implant as recited in claim 6, wherein the MTFN stent cover has a thickness ranging between approximately 5 μm and 10 μm.

8. An implant as recited in claim 1, wherein the MTFN stent cover comprises at least one hydrophilic surface having a water contact angle of approximately 40 degrees or less.

9. An implant as recited in claim 8, wherein the MTFN stent cover comprises at least one hydrophilic surface having a water contact angle of approximately 40 degrees.

10. An implant as recited in claim 1, wherein the fenestrations each have an elongate shape.

11. An implant as recited in claim 10, wherein the fenestrations comprise straight edges.

12. An implant as recited in claim 1, wherein the MTFN stent cover comprises:
a generally rectangular thin film sheet wrapped into a generally tubular shape having a longitudinal and radial direction;
wherein two longitudinal edges of the sheet at least meet to form said tubular shape; and
wherein the sheet has a compacted form with a first internal diameter and a deployed form with a second internal diameter larger than the first internal diameter.

13. An implant as recited in claim 12, wherein the implant is configured to be delivered into a blood vessel in the compacted form; wherein the implant is configured to be expanded to its deployed form at a treatment location within the blood vessel; and wherein the implant is configured to expand onto an internal surface of the blood vessel to at least contact said internal surface.

14. An implant as recited in claim 12, wherein the plurality of stent struts are configured to be disposed in a compressed form when constrained inside a catheter; wherein the plurality of stent struts are configured to automatically expand at the treatment site when not constrained inside said catheter; and wherein the MTFN stent cover is configured to expand with expansion of the stent struts.

15. A stent for treating arterial disease, comprising:
a tubular truss;
a sheet comprising thin film nickel titanium (NiTi) configured to surround the circumference of the truss;
the sheet having a compacted form having a first internal diameter and a deployed form having a second internal diameter larger than the first internal diameter;
wherein the stent is configured to be delivered into a blood vessel in the compacted form;
wherein the stent is configured to expand to its deployed form at a treatment location within the blood vessel; and
wherein the sheet is configured to expand onto an internal surface of the blood vessel to contact said internal surface, the sheet comprising a micro pattern of fenestrations;
wherein the fenestrations are sized to be small enough to substantially inhibit abluminal SMC migration through the sheet, while still allowing for intercellular communication through the sheet.

16. A stent as recited in claim 15, wherein the fenestrations are sized to have a maximum dimension of less than approximately 10 μm.

17. A stent as recited in claim 16, wherein the fenestrations are sized to have a maximum dimension of between approximately 5 μm and 10 μm.

18. A stent as recited in claim 15, wherein the thin film sheet has a thickness of less than approximately 50 μm.

19. A stent as recited in claim 18, wherein the thin film sheet has a thickness of less than approximately 10 μm.

20. A stent as recited in claim 19, wherein the thin film sheet has a thickness of ranging between approximately 5 μm and 10 μm.

21. A stent as recited in claim 15, wherein the sheet comprises at least one hydrophilic surface having a water contact angle of approximately 40 degrees or 10 less.

22. A stent as recited in claim 21, wherein the sheet comprises at least one hydrophilic surface having a water contact angle of approximately 40 degrees.

23. A stent as recited in claim 15, wherein the fenestrations comprise an elongate shape.

24. A stent as recited in claim 23, therein the fenestrations comprise straight edges.
